(19) United States
(12) Patent Application Publication
(10) Pub. No.: US 2010/0135965 A1
(43) Pub. Date: Jun. 3, 2010

(54) METHOD OF CHONDROGENIC DIFFERENTIATION FROM MESENCHYMAL STEM CELL, AND COMPOSITION COMPRISING CHONDROGENIC CELL DIFFERENTIATED USING THE METHOD TO TREAT DISEASE CAUSED BY CARTILAGE DAMAGE

(75) Inventors: Min-Suk Jeong, Jeongeup-si (KR); Moon-Youn Jung, Daejeon (KR); Seon-Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/471,570

(22) Filed: May 26, 2009

(30) Foreign Application Priority Data
Nov. 29, 2008 (KR) 10-2008-0120190

(51) Int. Cl. A61K 35/12; C12N 5/06; C12N 5/08

(52) U.S. Cl. 424/93.7; 435/377; 435/366

(57) ABSTRACT

Provided are a method of chondrogenic differentiation from mesenchymal stem cells and a composition comprising chondrogenic cells differentiated using the method to treat diseases caused by cartilage damage. In the method, a centrifugal force is applied to human mesenchymal stem cells to be differentiated into chondrogenic cells. The chondrogenic differentiation may be achieved at moderate cost without using expensive cytokines or growth factors by periodically applying only a centrifugal force. According to the method of chondrogenic differentiation from mesenchymal stem cells in the present embodiment, chondrogenic cells may be also differentiated from human mesenchymal stem cells.

### CROSS-REFERENCE TO RELATED APPLICATIONS

This U.S. non-provisional patent application claims priority under 35 U.S.C. § 119 of Korean Patent Application No. 10-2008-0120190, filed on Nov. 29, 2008, the entire contents of which are hereby incorporated by reference.

### BACKGROUND OF THE INVENTION

The present invention disclosed herein relates to a method of chondrogenic differentiation from mesenchymal stem cells, and a composition comprising chondrogenic cells differentiated using the method to treat disease caused by cartilage damage.

Stem cells are cells before being differentiated into individual cells constituting a tissue. Stem cells refer to cells that can proliferate infinitely in an undifferentiated state and have the potential to differentiate into various types of tissue cells through specific differentiation stimuli.

According to their differentiation potential, stem cells can be divided into embryonic stem cells (ESCs) and adult stem cells (ASCs) (also known as tissue-specific stem cells). ESCs are isolated from the inner cell mass (ICM) (the portion that develops into a fetus) in the very early blastocyst stage after an egg is fertilized and before the fertilized egg is implanted in the endometrium, and are cells with the potential to differentiate into cells of all tissue types.

Alternatively, tissue specific stem cells are organ-specific stem cells, which appear in the embryogenesis stages to form the various embryonic organs, and their differentiation potency is generally limited to constituting specific types of tissue cells (multipotent). Representative tissue-specific stem cells include hematopoietic stem cells in bone-marrow and mesenchymal stem cells that are differentiated into connective tissue cells (with the exclusion of blood cells). Hematopoietic stem cells are differentiated into various blood cells such as erythrocytes and leucocytes, and mesenchymal stem cells are differentiated into osteoblasts, chondroblasts, adipocytes, and myoblasts.

Recently, successful isolation of human embryonic stem cells, their clinical application has become a major area of interest. The field of cell replacement therapy has an especially high level of interest in stem cells, hoping to apply them as cell suppliers.

Cytokines or growth factors are involved in the differentiation of mesenchymal stem cells into chondrogenic cells, and further into chondrocytes (that is, chondrogenic differentiation). Although the precise mechanism has yet to be elucidated, TGF-β (transforming growth factor beta), IGF (insulin-like growth factor), BMP (bone morphogenic protein), FGF (fibroblast growth factor), and so on are known to play an important role in differentiation into chondrocytes. However, growth factors such as TGF-β are not only expensive per se, but rather accelerate aging of cells, accompanied by a decrease in cell viability, limiting their clinical use.

While chondrogenic differentiation methods through physical and mechanical stimuli have been reported, typical technologies have generally focused on animal (and not human) mesenchymal stem cell research. Because the differentiation of human mesenchymal stem cells is more difficult than that of animal mesenchymal stem cells, there are limitations in directly applying typical results from animal mesenchymal stem cells to human mesenchymal stem cells. In addition, it is almost impossible to apply and inject a composition comprising chondrocytes differentiated from animal mesenchymal stem cells directly to humans as a composition for treating cartilage damage.

### SUMMARY OF THE INVENTION

The present invention provides a method of chondrogenic differentiation from mesenchymal stem cells at moderate cost, without using expensive cytokines or growth factors.

The present invention also provides a composition comprising chondrogenic cells applicable to humans for treating damage caused by cartilage damage.

Embodiments of the present invention provide methods for differentiating mesenchymal stem cells into chondrogenic cells, including conducting a monolayer culture of mesenchymal stem cells, three-dimensionally culturing the monolayer-cultured mesenchymal stem cells, and applying centrifugal force to the three-dimensionally cultured-mesenchymal stem cells to be differentiated into chondrogenic cells.

In some embodiments, the mesenchymal stem cells are preferably human mesenchymal stem cells.

In other embodiments, the mesenchymal stem cells may be derived from human embryos, adult tissue, or bone marrow.

In still other embodiments, the applying of the centrifugal force to the three-dimensionally cultured-mesenchymal stem cells may be preferably performed at about 10 to about 200 G-force.

In even other embodiments, the applying of the centrifugal force to the three-dimensionally cultured-mesenchymal stem cells may be preferably performed for about 10 to about 30 minutes every day for 2 to 4 consecutive weeks.

The three-dimensional culture may be conducted by using alginate beads or PGA (Poly(glycolic acid)) scaffolds, or a pellet culture may be used. In order to produce a three-dimensional structure for the 3D-culture, collagen, gelatin, chitosan, hyaluronic acid, dextran or poly(lactic acid) may be used.

According to the present invention in order to achieve the other objects, a composition for treating diseases caused by cartilage damage comprises chondrogenic cells differentiated by the methods.

The diseases caused by cartilage damage may include degenerative arthritis, rheumatic arthritis, fracture, damage to muscular tissue, plantar fascitis, humerus epicondylitis, myositis ossificans, nonunion of fracture, or joint injury by an external wound.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a perspective view illustrating applying centrifugal force to mesenchymal stem cells for chondrogenic differentiation according to an embodiment of the present invention;

FIG. 2A is a picture illustrating mesenchymal stem cells histologically stained with safranin-O, in the control group without applying centrifugal force in Experimental Example 3-2;

FIG. 2B is a picture illustrating mesenchymal stem cells histologically stained with safranin-O, in the group subjected to centrifugal force in Experimental Example 3-1;

FIG. 3A is a picture illustrating mesenchymal stem cells immunohistologically analyzed in the control group without applying centrifugal force in Experimental Example 3-2;

FIG. 3B is a picture illustrating mesenchymal stem cells immunohistologically analyzed in the group subjected to centrifugal force in Experimental Example 3-1; and

FIG. 4 is an electrophoresis picture illustrating the results of RT-PCRs of mesenchymal stem cell RNAs in the group subjected to centrifugal force and the control group, with primers for type II collagen and aggrecan.

### DETAILED DESCRIPTION OF PREFERRED EMBODIMENTS

The present invention will be described below in more detail. The present invention may, however, be embodied in different forms and should not be construed as limited to the embodiments set forth herein. Rather, these embodiments are provided so that this disclosure will be thorough and complete, and will fully convey the scope of the present invention to those skilled in the art. In the present invention, the research is focused on chondrogenic differentiation of human mesenchymal stem cells, but it is apparent to those skilled in the art that the research is applicable to animal mesenchymal stem cells, which are more easily differentiated than human mesenchymal stem cells.

First, mesenchymal stem cells are prepared in order to accomplish a chondrogenic differentiation from the mesenchymal stem cells. The mesenchymal stem cells are preferably human, and may be derived from human embryos, adult tissue, or bone marrow. The mesenchymal stem cells may be obtained from a stem cell bank, or extracted and cultured directly from a patient's bone marrow, and so on.

A monolayer culture (Two-dimensional culture) of the prepared mesenchymal stem cells is conducted, for example, in a culture dish. The monolayer culture is performed by placing the mesenchymal stem cells into a growth medium supplemented with bovine fetal serum and antibiotics in a 150 mm culture dish at a concentration of about 1×10⁶ cells. The culture may be incubated in a 5% CO₂ incubator at 37° C. by attaching the cells to the culture dish. The medium may be exchanged once every three days and a subculture may be performed once every week. The number of mesenchymal stem cells suitable for a three-dimensional culture may be secured through these monolayer cultures.

After the monolayer culture is completed, the mesenchymal stem cells are detached from the culture dish to conduct a three-dimensional culture. The three-dimensional culture may include a pellet culture conducted in pellet form or a culture using alginate beads or PGA (acronym for Polyglycolide and an aliphatic polyester which is a kind of poly(a-hydroxy acid)) scaffold. The three-dimensional forms are manufactured, placed into the chondrogenic differentiation medium, and then put in the incubator. The chondrogenic differentiation medium may be exchanged, for example, once every three days.

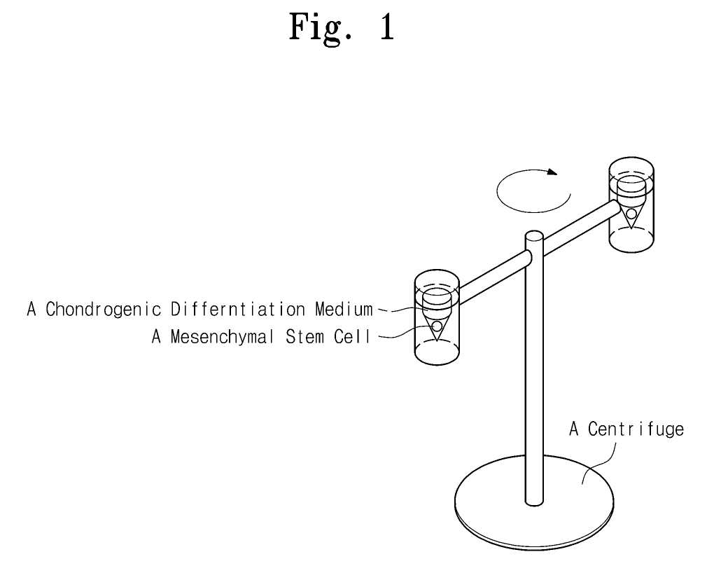

The three-dimensional structure of the mesenchymal stem cells in the chondrogenic differentiation medium is withdrawn from the incubator, placed into a centrifuge illustrated in FIG. 1, and then rotated preferably at about 10 to about 200 G-force to apply centrifugal force to the mesenchymal stem cells. These processes may be performed for about 10 to about 30 minutes every day for 2 to 4 consecutive weeks. Through these processes, cells having characteristics of chondrogenic cells or chondrocytes may be differentiated from mesenchymal stem cells. Furthermore, chondrocytes may be differentiated.

Whether chondrogenic cells or chondrocytes are differentiated may be determined by using safranin-O and/or an immunohistological analysis to identify whether GAG protein and type II collagen protein have color development, and whether lacunas (characteristics of chondrocytes) are formed.

Whether chondrogenic cells or chondrocytes are differentiated may be also determined by identifying the expressions of type II collagen (a marker for chondrogenic differentiation) and aggrecan gene.

Chondrogenic cells or chondrocytes differentiated by these methods may be used as a composition for treating diseases caused by cartilage damage. The PGA or alginate has the property of being degraded and absorbed spontaneously in vivo. Thus, chondrogenic cells or chondrocytes differentiated by using the PGA scaffolds or alginates may be used as a composition in which the PGA or alginates are included.

For example, mesenchymal stem cells, which are derived from a born marrow of a patient who has cartilage injuries, can be cultured and applied by a centrifugal force to be differentiated to chondrogenic cells or chondrocytes. These chondrogenic cells or chondrocytes can be injected into the cartilage of the patient as a composition for medical cure.

Furthermore, an artificial joint may be manufactured by differentiating mesenchymal stem cells of the present invention into chondrogenic cells or chondrocytes on a joint-shaped three-dimensional support matrix.

Cartilage diseases, which mesenchymal stem cells produced by the method of the present invention may treat by using fixed biodegradable polymers, include, but are not limited to, degenerative arthritis, rheumatic arthritis, fracture, damage to muscular tissue, plantar fascitis, humerus epicondylitis, myositis ossificans, nonunion of fracture, or joint injury by external wound.

### Experimental Example 1

### Monolayer Culture

Human mesenchymal stem cells were purchased from LONZA (USA). The mesenchymal stem cells were attached to the bottom of a culture dish and a mesenchymal stem cell growth medium MSCGM bullet kit (LONZA, USA) was added to conduct a monolayer-culture. The culture dish was incubated in a 5% CO₂ incubator at 37. The medium was exchanged once every three days, and the number of cells suitable for a three-dimensional culture was secured through a subculture performed once every week.

### Experimental Example 2

### Three-Dimensional Culture

The mesenchymal stem cells incubated in Experimental Example 1 were treated with 0.05% trypsin-EDTA (ethylenediaminetetraacetate) and detached from the culture dish.

One group of the mesenchymal stem cells was suspended in a 2% alginate solution at a concentration of about 2×10⁶ cells/d, and cell/alginate suspensions were slowly dropped into a 102 mM CaCl₂ solution and left still for 10 minutes to form spherical beads.

Another group of the mesenchymal stem cells, that was detached by the method for inoculation of the mesenchymal stem cells into PGA scaffolds, was incubated in a CO₂ incubator at a concentration of about 5×10⁶ cells/ml for about 4 hours and directly injected into the PGA scaffolds.

Each of the three-dimensional structures thus manufactured was placed in a chondrogenic differentiation medium [high glucose DMEM (hyclone, USA), ITS (ITS LIQUID MEDIA SUPPLEMENT) (Sigma, USA), 50 μg/ml ascorbic acid 2-phosphate, 100 mM dexamethasone (Sigma, USA), 40 μg/ml proline, 1.25 mg/ml BSA (bovine serum albumin) (Sigma, USA), and 100 μg/ml sodium pyruvate (Sigma, USA)] and incubated in the same incubator as in Experimental Example 1.

### Experimental Example 3-1

### Group Subjected to Centrifugal Force

Chondrogenic differentiation was induced by applying centrifugal force to some of the alginate beads and the PGA scaffolds including the mesenchymal stem cells in Experimental Example 2. Centrifugal force of about 100×G-force was applied to the mesenchymal stem cells in the group subjected to centrifugal force once every day for 2 weeks and the incubation was performed by exchanging the medium in a CO₂ incubator at 37° C. once every three days.

### Experimental Example 3-2

### Control Group

The incubation was performed by exchanging the medium in a 5% CO₂ incubator at 37° C. once every three days without applying centrifugal force to the other alginate beads and the PGA scaffolds including the mesenchymal stem cells in Experimental Example 2.

### Experimental Example 4

### Immunohistological Analysis 1

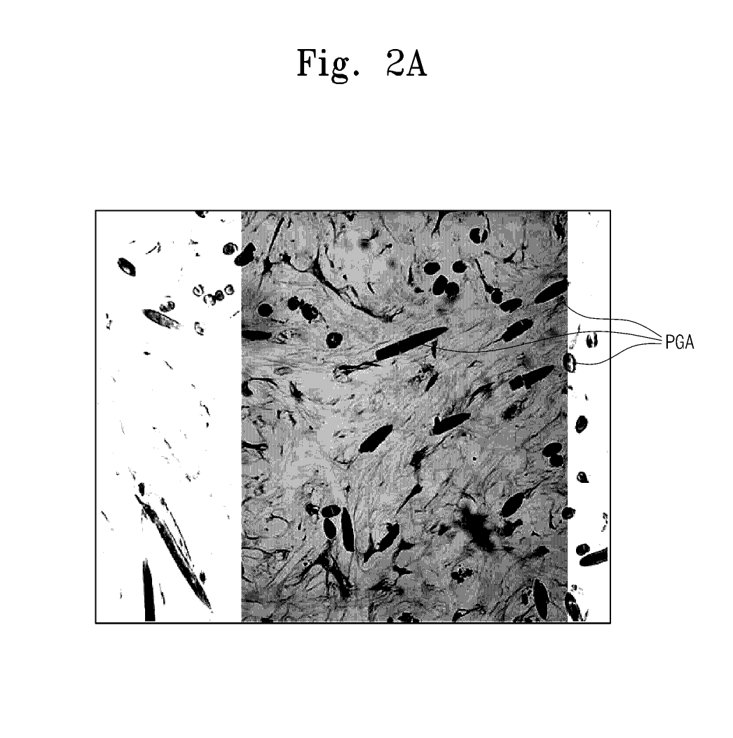

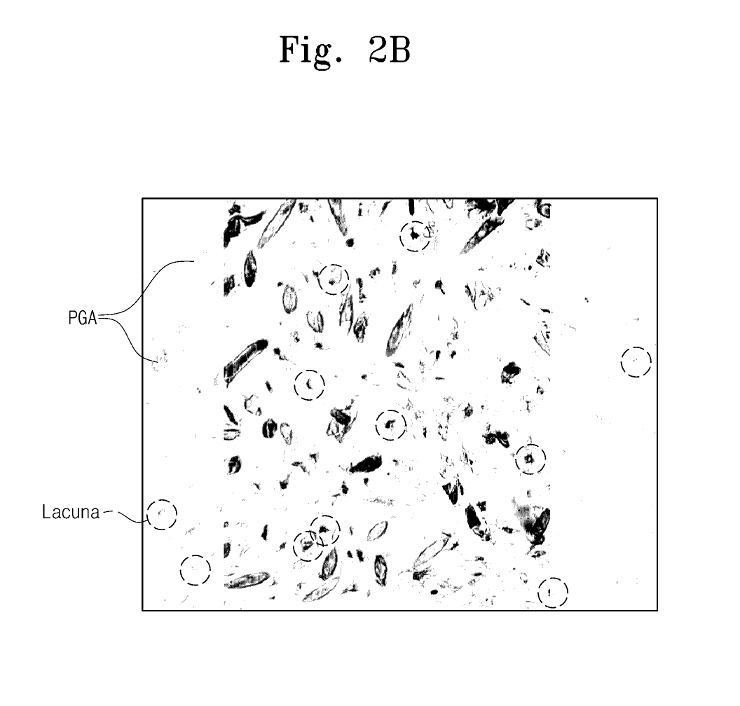

Each of the mesenchymal stem cells incubated in Experimental Examples 3-1 and 3-2 and included in the PGA scaffolds was washed respectively with PBS (Phosphate Buffer Saline) and fixed in a 10% formalin solution. Each of the fixed samples was dehydrated and embedded in paraffin blocks. After the blocks were cut into 4 μm sections, some of the sections were stained with safranin-O, and microscopic photos (depicted respectively in FIGS. 2a and 2b) were captured. GAG (glycosaminoglycan) protein, a marker for expression of aggrecan genes which are characteristic of chondrogenic cells, is stained red with safranin-O. Comparing the photo of the mesenchymal stem cells from the group subjected to centrifugal force in FIG. 2b with that of the mesenchymal stem cells of the control group (in FIG. 2a) that were incubated in a static state without being subjected to centrifugal force, it is apparent from the increased number of red-stained parts in FIG. 2b that the expression of GAG protein increased, and the presence of lacuna was frequently observed. Thus, it can be noted that chondrogenic cells or chondrocytes were differentiated from mesenchymal stem cells by applying centrifugal force. It can also be noted that differentiation of chondrogenic cells was not observed in FIG. 2a and the cells were present as mesenchymal stem cells. The dark portions in the FIGS. 2a and 2b photos indicate PGAs.

### Experimental Example 5

### Immunohistological Analysis 2

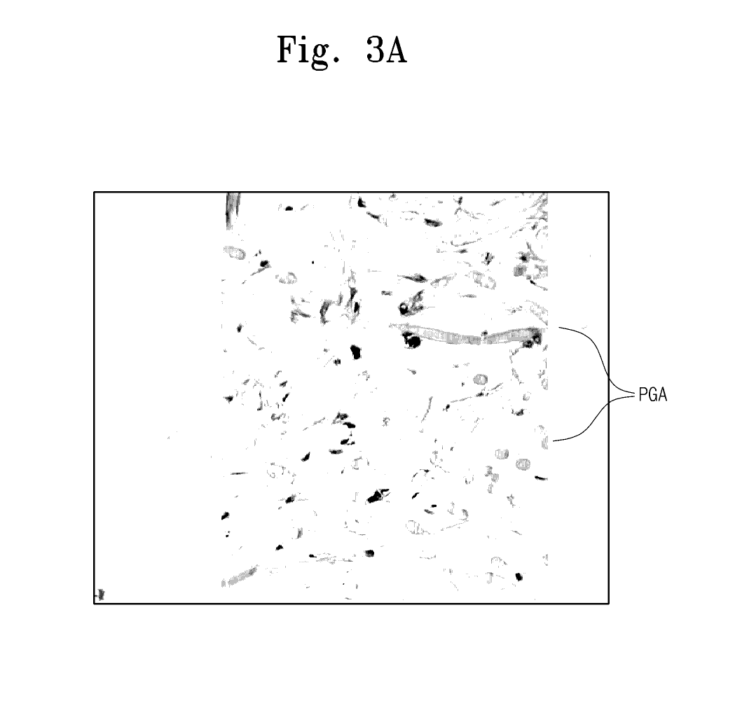

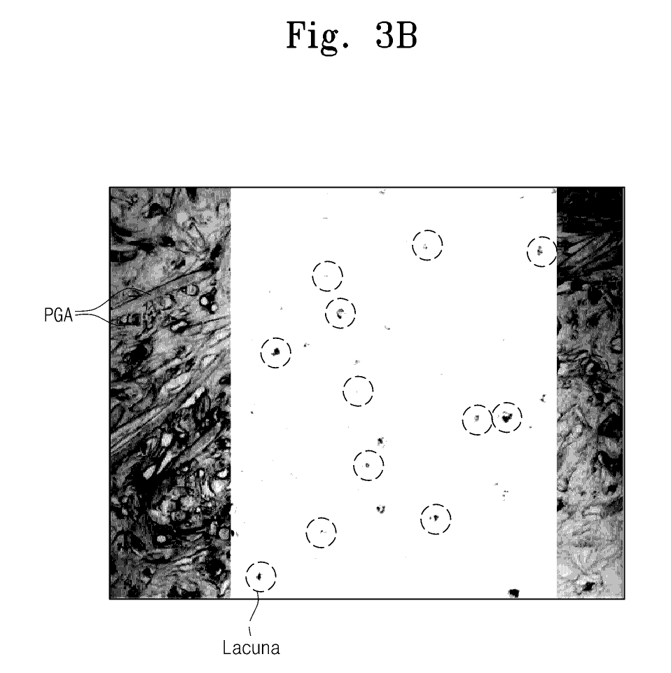

In order to identify the expression of type II collagen, a marker for chondrogenic differentiation, intracellular peroxidase was removed from the others of the paraffin block sections in Experimental Example 4 with a 3% H₂O₂ solution, treated with pepsin, and then reacted with a 1% BSA (bovine serum albumin) solution to inhibit a non-specific reaction. Mouse anti-human type II collagen antibodies were used to carry out a reaction, and an avidin-biotin reaction was used to amplify the signal. The type II collagen protein was stained brown with DAB (diaminobenzidine) and photographed with a microscope. The photos are depicted respectively in FIGS. 3a and 3b. Comparing the picture of the mesenchymal stem cells in the group subjected to centrifugal force in FIG. 3b with that of the mesenchymal stem cells incubated in a static state without applying centrifugal force in the control group in FIG. 3a, it is apparent from the increased number of brown-stained parts in FIG. 3b that the expression of type II collagen protein increased, and the presence of lacuna was frequently observed. Thus, it can be noted that chondrogenic cells or chondrocytes were differentiated from mesenchymal stem cells by applying centrifugal force. It can also be noted that differentiation of chondrogenic cells was not observed in FIG. 3a and the cells were present as mesenchymal stem cells.

### Experimental Example 6

### Identification of Chondrogenic Differentiation Marker RNA Expression

In order to identify a degree of chondrogenic differentiation of mesenchymal stem cells as a result of Experimental Example 3-1, the expressions of type II collagen (a marker for chondrogenic differentiation) and aggrecan gene were identified through RT-PCT (Reverse Transcriptase Polymerase Chain Reaction).

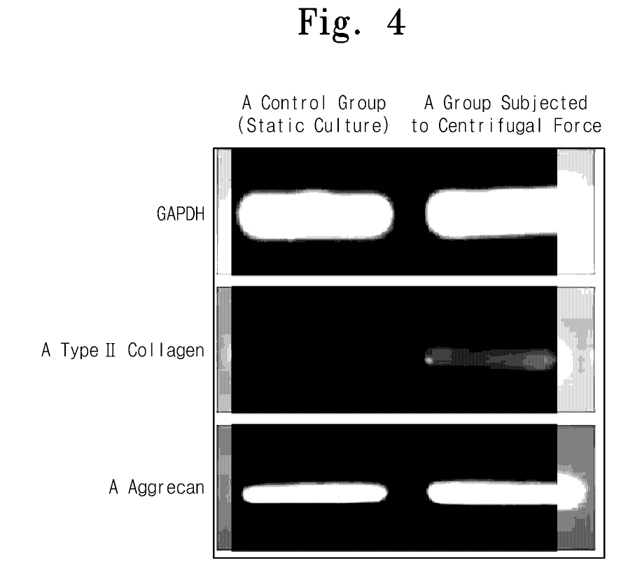

Mesenchymal stem cells were isolated from alginate beads by using 55 mM sodium citrate, and then the total RNA was isolated by using a Trizol solution (invitrogen). For 2 μg of the isolated RNA, AMV reverse transcriptase (Roche) was used to synthesize cDNA, and then the cDNA was amplified through a PCR reaction by using gene-specific primers (type II collagen and aggrecan) listed in Table 1. A primer for GAPDH, a gene whose expression amount is constant under any conditions, was used as a control group. The PCR product was electrophoresized by a 1.5% agarose gel, and the pictures were shown in FIG. 4.

TABLE 1

| Gene | Primer | Base Sequence (Human) |
|---|---|---|
| Type II | Sense | 5′-CTCCTGGAGCATCTGGAGAC-3′ |
| collagen | Antisense | 5′-ACCACGATCACCCTTGACTC-3′ |
| Aggrecan | Sense | 5′-TGAGTCCTCAAGCCTCCTGT-3′ |
| | Antisense | 5′-CAGTGGCCCTGGTACTTGTT-3′ |
| GAPDH | Sense | 5′-GGTCATGAGTCCTTCCACGAT-3′ |
| | Antisense | 5′-GGTGAAGGTCGGAGTCAACGG-3′ |

Referring to FIG. 4, it can be seen that the areas of type II collagen and aggrecan in the group subjected to centrifugal force are larger than those of type II collagen and aggrecan in the control group, which illustrates that the expressions of type II collagen and aggrecan in the group subjected to centrifugal force are increased more than those of type II collagen and aggrecan in the control group. Therefore, it can be concluded that chondrogenic cells or chondrocytes were differentiated from mesenchymal stem cells by applying centrifugal force.

According to a method of chondrogenic differentiation from mesenchymal stem cells in the present embodiment, centrifugal force is applied to human mesenchymal stem cells to be differentiated into chondrogenic cells. The chondrogenic differentiation may be achieved at moderate cost by periodically applying only centrifugal force without using expensive cytokines or growth factors. According to a method of chondrogenic differentiation from mesenchymal stem cells in the present embodiment, chondrogenic cells may be also differentiated from human mesenchymal stem cells.

In addition, a composition for treating diseases caused by cartilage damage in the present embodiment comprises human chondrogenic cells differentiated by a method of chondrogenic differentiation from human mesenchymal stem cells, and it is possible to apply or inject the composition directly to humans.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A method of chondrogenic differentiation from mesenchymal stem cells, the method comprising:
culturing mesenchymal stem cells into a monolayer;
three-dimensionally culturing the monolayer-cultured mesenchymal stem cells; and
applying a centrifugal force to the three-dimensionally cultured mesenchymal stem cells to be differentiated into chondrogenic cells.

2. The method of claim 1, wherein the mesenchymal stem cells are human mesenchymal stem cells.

3. The method of claim 1, wherein the applying of the centrifugal force to the three-dimensionally cultured mesenchymal stem cells is performed at about 10 to about 200 G-force.

4. The method of claim 1, wherein the applying of the centrifugal force to the three-dimensionally cultured mesenchymal stem cells is performed for about 10 to about 30 minutes every day for 2 to 4 consecutive weeks.

5. The method of claim 1, wherein the mesenchymal stem cells are derived from human embryos, adult tissue, or bone marrow.

6. A composition for treating diseases caused by cartilage damage, comprising chondrogenic cells differentiated by conducting a monolayer culture of mesenchymal stem cells, three-dimensionally culturing the monolayer cultured mesenchymal stem cells, and applying a centrifugal force to the three-dimensionally cultured-mesenchymal stem cells to be differentiated into the chondrogenic cells.

7. The composition of claim 6, wherein the diseases caused by cartilage damage comprise degenerative arthritis, rheumatic arthritis, fracture, damage to muscular tissue, plantar fascitis, humerus epicondylitis, myositis ossificans, nonunion of fracture, or joint injury by an external wound.

8. The composition of claim 6, wherein the mesenchymal stem cells are human mesenchymal stem cells.

9. The composition of claim 6, wherein the applying of the centrifugal force to the three-dimensionally cultured mesenchymal stem cells is performed at about 10 to about 200 g-force.

10. The composition of claim 6, wherein the applying of the centrifugal force to the three-dimensionally cultured mesenchymal stem cells is performed for about 10 to about 30 minutes every day for 2 to 4 consecutive weeks.

11. The composition of claim 6, wherein the mesenchymal stem cells are derived from human embryos, adult tissue, or bone marrow.


SEQUENCE LISTING (6 sequences)

SEQ ID NO: 1 - DNA, 20 bp, Homo sapiens
ctcctggagcatctggagac

SEQ ID NO: 2 - DNA, 20 bp, Homo sapiens
accacgatcacccttgactc

SEQ ID NO: 3 - DNA, 20 bp, Homo sapiens
tgagtcctcaagcctcctgt

SEQ ID NO: 4 - DNA, 20 bp, Homo sapiens
cagtggccctggtacttgtt

SEQ ID NO: 5 - DNA, 21 bp, Homo sapiens
ggtcatgagtccttccacgat

SEQ ID NO: 6 - DNA, 21 bp, Homo sapiens
ggtgaaggtcggagtcaacgg